(19) United States
(12) Patent Application Publication
(10) Pub. No.: US 2006/0293595 A1
(43) Pub. Date: Dec. 28, 2006

(54) System and method for ultrasound pulse shaping and output power adjustment using multiple drive pulses

(76) Inventors: David W. Clark, BRIARCLIFF MANOR, NY (US); David S. Sherrill, Westford, MA (US)

Correspondence Address: PHILIPS INTELLECTUAL PROPERTY & STANDARDS, P.O. BOX 3001, BRIARCLIFF MANOR, NY, 10510, US

(21) Appl. No.: 10/569,252

(22) PCT Filed: Aug. 18, 2004
(86) PCT No.: PCT/IB04/51487

§ 371 Date: Feb. 21, 2006

Related U.S. Application Data
(60) Provisional application No. 60/498,000, filed on Aug. 26, 2003.

(51) Int. Cl. A61B 8/00

(52) U.S. Cl. 600/437

(57) ABSTRACT

The present invention provides a system and method for ultrasound pulse shaping and output power adjustment using multiple drive pulses. The multiple drive pulses are width modulated to provide the required output signal power and wave-shape characteristics. Using multiple width-modulated pulses provides control over power output that is capable of being varied much more rapidly than using a conventional voltage modulated drive pulse. Additionally, the multiple drive pulses provide better control over unwanted harmonics than does a single drive pulse. These two advantages allow multiple width modulated pulses to increase the capabilities in ultrasonic imaging devices, thereby, allowing for rapid switching between imaging techniques having widely disparate power requirements so that composite diagnostic images may be constructed combining the diagnostic benefits the various imaging techniques have to offer.

The present invention relates to acoustic waveform generation and specifically to ultrasound pulse shaping using multiple drive pulses.

Ultrasound imaging systems commonly in use generate and transmit ultrasound signals to map internal tissue typography, vascular fluid flow rates, and abnormalities. The systems typically incorporate several methods, or modes, of imaging, i.e. Brightness Mode (B-Mode), Harmonic, Spectral Doppler, and Color Flow.

Each imaging method has its characteristic uses and limitations. B-Mode imaging is typically used to image a “snapshot” of internal tissue and organs with high spatial resolution. Generally to achieve this degree of spatial resolution, short-duration ultrasound pulses are advantageous as are lower frequencies—lower frequency ultrasound having better penetration.

Color Flow imaging is primarily used to measure blood flow rates and detect abnormal and destructive turbulent flows within the cardiovascular system. Color Flow images are usually overlaid on to a B-Mode structural snapshot. However, the ultrasound properties necessary for proper Color Flow imaging differ from those used in B-Mode. Low ultrasound pulse repetition rates are desirable for slow-flowing veins, but for the faster flows found in the arteries and heart, higher ultrasound pulse repetition rates are necessary to properly avoid aliasing errors. The sensitivity necessary for Color Flow imaging requires higher ultrasound frequencies than commonly used for the deeper penetrating B-Mode scans. Additionally, Color Flow imaging uses higher-intensity power than B-Mode.

Harmonic imaging uses the harmonic frequencies produced when a transmitted fundamental frequency is reflected by tissues and other internal structures. Proper Harmonic imaging, thus, requires transmission of ultrasound fundamental frequencies without the associated harmonics, which would be confused with the reflected harmonics. Harmonic imaging makes use of narrowly tuned frequencies achievable through waveform shaping as disclosed in U.S. Pat. No. 5,833,614 “Ultrasonic Imaging Method and Apparatus for Generating Pulse Width Modulated Waveforms with Reduced Harmonic Response” issued to Dodd et al. and incorporated herein by reference in its entirety.

In all of these imaging methods, it is also desirable to control the power output of the emitted ultrasound pulse. Power output is reduced when imaging delicate tissues such as fetal tissue or to prevent over-heating of the transducer and patient-contact area thus preventing burns to the patient and damage to the ultrasound transducer. One method for controlling power output commonly in use consists of systems to regulate voltage, either automatically or manually, to the ultrasound transducer. However, this power output control method has a relatively slow response time—on the order of hundreds of milliseconds—and may compromise image quality, therefore, voltage modulation is not appropriate in situations where the power level needs to be rapidly varied without loss of image quality, as in the case of Color Flow/B-Mode combination scans.

An object of the present invention is to provide a system and method for controlling power output having faster response time than obtained with conventional voltage modulation method.

An additional object of the present invention is to provide a system and method of shaping the output waveform in order to reduce harmonics-induced transducer heating and provide a more versatile imaging system.

Another object of the present invention is to provide a system and method of shaping the output waveform allowing power output characterization as required for medical-use certifications that is less complex and time consuming than needed by prior art single pulse width modulation.

The present invention provides a system and method for ultrasound pulse shaping and output power adjustment using multiple Pulse Width Modulated drive pulses. Generally, a drive pulse is a square wave characterized by a duration, amplitude and frequency. These drive pulse characteristics directly affect the shape—frequency, amplitude, waveform, etc.—of the output signal. Thus, by varying the input pulse widths, the output signal can be shaped to meet the needs of most any situation.

The present invention uses multiple full amplitude drive pulses of varying durations and frequencies to create a desired output signal.

These and other features, aspects, and advantages of the present invention will become better understood with regard to the following description, appended claims, and accompanying drawings wherein:

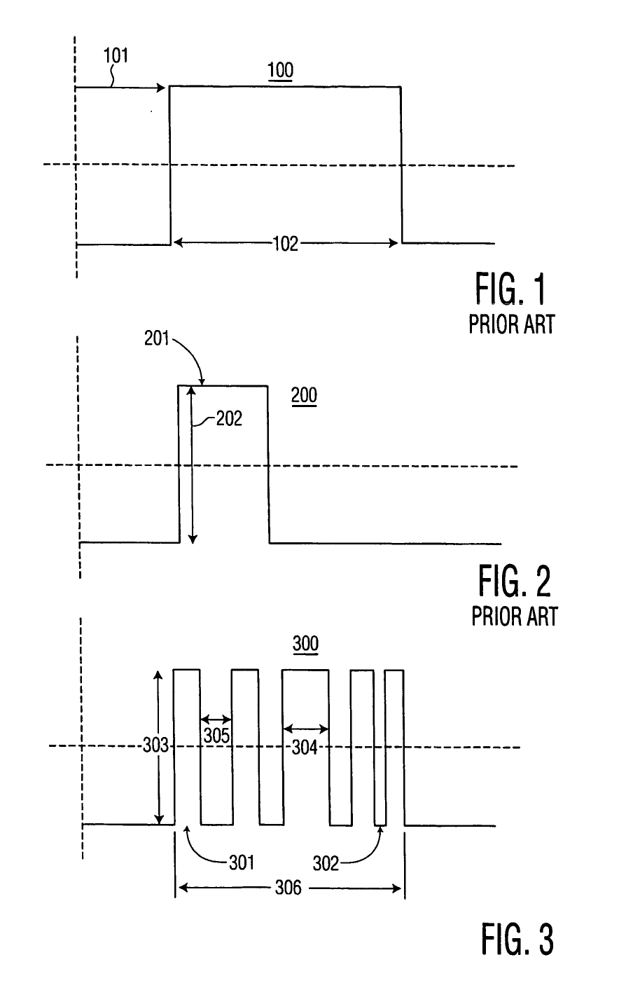

FIG. 1 is a graphical representation of a typical drive pulse;

FIG. 2 is a graphical representation of a prior art pulse width modulation of the drive pulse of FIG. 1;

FIG. 3 is a graphical representation of a pulse width modulation of the drive pulse of FIG. 1 in accordance with an embodiment of the present invention;

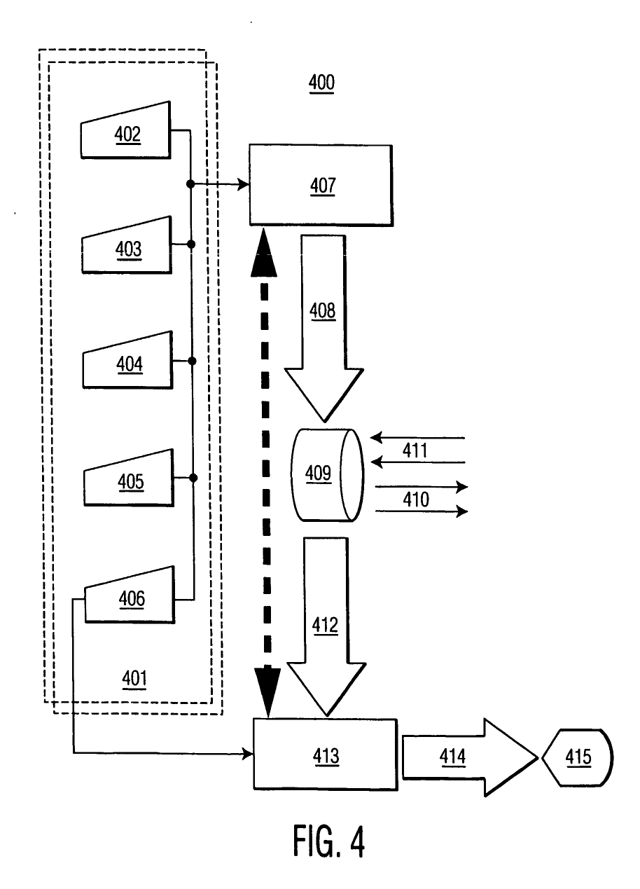

FIG. 4 is a schematic representation of an ultrasound imaging system in accordance with the present invention; and

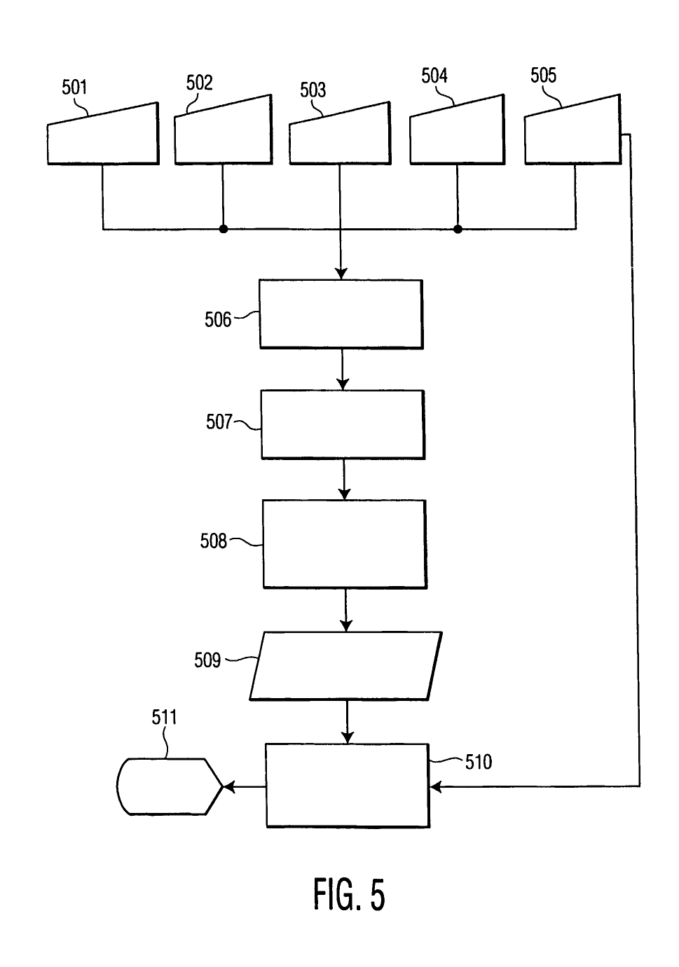

FIG. 5 is a procedural representation of the ultrasound imaging system of FIG. 4 in accordance with the present invention.

With reference to FIGS. 1, 2 and 3, a typical drive pulse 100 has a duration 102 and amplitude (power) 101 approximately equal to the desired output signal; meaning that if a lower power output signal is desired, the amplitude 101 of the drive pulse 100 would simply be decreased, resulting in a lower amplitude output signal. This method works fine when rapid amplitude fluctuations of the output signal are not required. However, as previously mentioned amplitude modulation is slow (˜200 ms).

Pulse Width Modulation (PWM) 200 solves this speed problem. The PWM method can switch on and off at a rate of mere microseconds—easily capable of meeting the rapid switching requirements for accurate Color Flow/B-Mode combination scans. PWM 200 relies on variable duration drive pulses 201 to achieve a desired power level from the output signal. The amplitude 202 of the drive pulse 201 remains at a constant value while the duration (or width) 201 is varied. Thus over the duration of the output signal, the total power averages out to less than full power. However, this technique creates some new problems. With this method, increased harmonics are produced.

The increased harmonics actually have two harmful effects. First, output signal harmonics direct power towards unusable frequencies and away from the fundamental frequency, increasing the overall energy needed to be transmitted to the patient in order to achieve a proper ultrasonic image. Second, in Harmonic Imaging, output signal harmonics interfere with the ultrasound imaging system's ability to detect the harmonics produced by the reflection of the output fundamental frequency by the internal tissues and structures of the patient. Additionally, ultrasound devices must be certified for use as medical devices; this certification requires that an output characterization be provided. Generally, in Voltage modulation, this characterization procedure is simple and straightforward, however in PWM systems, the procedure is more complex and time consuming since all possible input pulse widths need to be individually tested and characterized.

The method of the present invention entails multi-Pulse Width Modulation (m-PWM) 300. This method solves both the slow amplitude modulation rate issue and the production of output signal harmonics. In m-PWM, as shown by FIG. 3, multiple pulses 301 are generated each having constant amplitude 303 and a variable duration 304. These multiple pulses 301 are separated by pauses 302—also having variable durations 305. The group of pulses 301 and pauses 302 has a total duration 306 approximately equal to the actual duration of the output signal, but a reduced total power. The output signal produced with m-PWM would also have a significantly reduced harmonic output. As in the single PWM method, m-PWM can efficiently be switched on and off very rapidly. Additionally, by considering the band limiting effects of the pulsing electronics, the transmission line and the transducer itself, pulses can be chosen such that the acoustic pulse produced is substantially the same as an acoustic pulse produced using voltage modulation. Consequently, the spectral characteristics of the pulse are substantially the same as the voltage modulated pulse, and output characterization is no more complicated than for voltage modulation.

An embodiment of the present invention, as shown in FIG. 4, is a medical diagnostic imaging system 400, which incorporates the m-PWM method according to the present invention. In the present embodiment, the medical diagnostic imaging system 400 provides the user with an interface for specifying particular output signal characteristics 401 such as frequency 402, pulse duration 403, waveform 404, i.e. Sawtooth, Square, Sinusoidal, etc., output power 405, and imaging mode 406, i.e. B-Mode, Harmonic, Spectral Doppler, and Color Flow. The imaging mode 406 will also be used by a signal processor 413 to select the proper method for use in processing the return signal 412. The output signal characteristics 401 are forwarded to the signal generator 407. The signal generator 407 applies the specified output signal characteristics 401 to internal algorithms to produce a drive pulse train 408, which, when applied to the ultrasound transducer 409, will result in an output signal 410 having substantially similar characteristics as the user specified characteristics 401. The Signal generator may either employ a dedicated processor or utilize the signal processor 413 for executing the internal algorithms.

The output signal 410, emitted by the ultrasound transducer 409, impinges on and reflects off of various corporeal structures (not shown) resulting in a return signal 411. The return signal 411 is detected by an ultrasound receiver, which may either be an element and function of the ultrasound transducer 409 or an entirely separate unit. The return signal data 412 is transferred to the signal processor 413, which processes the return signal data 412 and produces image data 414, which are then transferred to a display apparatus 415. The display apparatus 415, may be any of the following: video display, printer, etc. additionally the display apparatus 415 may instead be replaced or supplemented by a data storage device, i.e. RAM, magnetic media, optical media, etc.

Referring to FIG. 5, an embodiment of the present invention begins with steps 501-505, in which the operator selects various options to set output signal characteristics—step 501 sets Frequency, step 502 sets Pulse Duration, step 503 sets Waveform, step 504 sets Output Power and step 505 sets Imaging Mode. Collectively, the output signal characteristics are forwarded to a processor, which subsequently performs step 506. In step 506, the processor uses the settings from steps 501-505 to determine the required drive pulse train characteristics that will yield an output ultrasound signal having the characteristics set in steps 501-505. Step 507 generates a drive pulse train having the characteristics determined in step 506 and applies the drive pulse train to an ultrasound transducer. Subsequently, step 508 transmits an output ultrasound signal directed towards a body region to be imaged. The transmitted output ultrasound signal is reflected by various tissues and body structures and the resulting imaging signal is detected in step 509. The process continues with step 510, in which the detected imaging signal is processed and analyzed based on the Imaging Mode setting of step 505. The operation ends with step 511, where the processed and analyzed image signal of step 510 is displayed in a user-interpretable manner, preferably on a video display as a graphical representation of the bodily region being imaged.

The described embodiments of the present invention are intended to be illustrative rather than restrictive, and are not intended to represent every embodiment of the present invention. Various modifications and variations can be made without departing from the spirit or scope of the invention as set forth in the following claims both literally and in equivalents recognized in law.

1. A method for controlling the output power of a signal produced by an ultrasound transducer, comprising the steps of:
producing an input drive pulse train consisting of at least two drive pulses having a fixed amplitude separated by a pause, wherein each of said at least two drive pulses and said pause have adjustable durations;
determining a value for said adjustable durations; and
providing a transducer configured to accept said input drive pulse train and emit an ultrasonic output signal from said input drive pulse train, wherein characteristics of said ultrasonic output signal are determined by characteristics of said input drive pulse train.

2. A method as in claim 1, wherein each of said adjustable durations may have different values.

3. A method as in claim 1, wherein said input pulse train is generated by a signal generator configured to determine said durations necessary to produce said ultrasonic output signal.

4. A method as in claim 1, wherein said drive pulses produced by said signal generator is of a type selected from the group consisting of: Sawtooth, Square and Sinusoidal.

5. A method as in claim 1, wherein said durations are adjusted according to values determined by said signal generator such that said ultrasound transducer produces said ultrasonic output signal having an overall reduced output power.

6. A method as in claim 1, wherein said durations are adjusted according to values determined by said signal generator such that said ultrasound transducer produces said ultrasonic output signal substantially free of harmonic frequencies.

7. A method as in claim 1, wherein said method is performed by a medical diagnostic system.

8. A method as in claim 1, further comprising the step of providing a plurality of imaging modes selected from the group consisting of: B-Mode, Color Flow, Harmonic and Spectral Doppler, said plurality of imaging modes are performed substantially simultaneously by rapidly repeating a scan line for each of said plurality of imaging modes before proceeding to a subsequent scan line.

9. An ultrasound imaging system with controllable output power, comprising:
a signal generator for generating an input drive pulse train, wherein said drive pulse train is comprised of at least two drive pulses having fixed amplitudes and adjustable pulse durations;
a transducer configured to accept said input drive pulse train and emit an ultrasonic output signal from said input drive pulse train, wherein characteristics of said ultrasonic output signal are determined by characteristics of said input drive pulse train;
a user interface configured to allow a user to adjust output signal values for said ultrasonic output signal; and
a processor configured for calculating and setting drive pulse values for said input drive pulse train characteristics, wherein said drive pulse values are calculated by said processor to produce said ultrasonic output signal having said user-adjusted output signal values.

10. The ultrasound imaging system as in claim 9, wherein said signal generator is configured to generate Sawtooth, Square, and Sinusoidal waveforms, having frequencies in the range from 20 KHz to 100 Mhz.

11. The ultrasound imaging system as in claim 9, wherein said input drive pulse durations are adjusted such that said ultrasound transducer generates said ultrasonic output signal having an overall reduced output power.

12. The ultrasound imaging system as in claim 9, wherein said input drive pulse durations are adjusted such that said ultrasound transducer generates said ultrasonic output signal substantially free of harmonic frequencies.

13. The ultrasound imaging system as in claim 9, wherein said processor is additionally configured to analyze reflected ultrasonic signals and produce a user-interpretable representation of the reflected ultrasonic signals.

14. The ultrasound imaging system as in claim 9, wherein said system is configured to provide a plurality of imaging modes selected from the group consisting of: B-Mode, Color Flow, Harmonic and Spectral Doppler, said plurality of imaging modes are performed substantially simultaneously by rapidly repeating a scan line for each of said plurality of imaging modes before proceeding to a subsequent scan line.

15. A system for controlling the output power of a signal produced by an ultrasound transducer, comprising:
means for producing an input drive pulse train consisting of at least two drive pulses having a fixed amplitude separated by a pause, wherein each of said at least two drive pulses and said pause have adjustable durations;
means for determining a value for said adjustable durations; and
means for providing a transducer configured to accept said input drive pulse train and emit an ultrasonic output signal from said input drive pulse train, wherein characteristics of said ultrasonic output signal are determined by characteristics of said input drive pulse train.

16. A system as in claim 15, wherein each of said adjustable durations may have different values.

17. A system as in claim 15, wherein said input pulse train is generated by a signal generator configured to determine said durations necessary to produce said ultrasonic output signal.

18. A system as in claim 15, wherein said drive pulses produced by said signal generator is of a type selected from the group consisting of: Sawtooth, Square and Sinusoidal.

19. A system as in claim 15, wherein said durations are adjusted according to values determined by said signal generator such that said ultrasound transducer produces said ultrasonic output signal having an overall reduced output power.

20. A system as in claim 15, wherein said durations are adjusted according to values determined by said signal generator such that said ultrasound transducer produces said ultrasonic output signal substantially free of harmonic frequencies.

21. A system as in claim 15, wherein said system is a medical diagnostic system.

22. A system as in claim 15, further comprising a means for providing a plurality of imaging modes selected from the group consisting of: B-Mode, Color Flow, Harmonic and Spectral Doppler, said plurality of imaging modes are performed substantially simultaneously by rapidly repeating a scan line for each of said plurality of imaging modes before proceeding to a subsequent scan line.
